(19) United States
(12) Patent Application Publication
(10) Pub. No.: US 2005/0274817 A1
(43) Pub. Date: Dec. 15, 2005

(54) Perfume gel composition

(76) Inventor: Huib Maat, Strawberry Hill (GB)

Correspondence Address: Paul Grandinetti;Levy & Grandinetti, 1725 K Street N W, Suite 408, Washington, DC, 20006-1419, US

(21) Appl. No.: 10/506,193

(22) PCT Filed: Mar. 6, 2003
(86) PCT No.: PCT/GB03/00930

§ 371 Date: Jul. 5, 2005

(30) Foreign Application Priority Data
Mar. 6, 2002 (GB) 0205220.7
Aug. 14, 2002 (GB) 0218892.8

(51) Int. Cl. A61K 7/46; A61L 9/04; A24F 25/00

(52) U.S. Cl. 239/34; 239/54; 239/55

(57) ABSTRACT

There is described a gel comprising from 3 to 80% w/w of perfume base, characterised in that the gel element comprises a substantially anhydrous thermally reversible gel. There is also described an air freshener, a lavatory cleanser/deodoriser and/or an insecticide/deodoriser comprising such a gel element.

The present invention relates to novel perfumed gel compositions, methods for their preparation and air freshening device related thereto.

Most existing air freshener gels are termed “hydrogels”, that is they are colloidal dispersions comprising droplets of fragrance distributed within a hydrocolloid matrix. The hydrocolloid used to prepare such gels may typically be for instance carageenan, alginate, carboxymethylcellulose (CMC), gelatin or gellan glum. The fragrance droplets may be stabilised by the addition of a surface active agent. In compositions in which the gel element is opaque the level of surface active agent is relatively low, for example less than 1% w/w based on the total composition. In compositions in which the gel element is transparent, for example, gel elements incorporating gellan gum or gelatin, the level of surface active agent is higher, for example, greater than 8% w/w, so that the fragrance forms a microemulsion within the hydrocolloid matrix. Hydrogels typically contain a maximum of 10% wv/w of a perfume base. Therefore, one particular disadvantage of such hydrogels is that a relatively large amount of the gel composition is required to adequately freshen the surrounding atmosphere. Thus, for example, it is usual for the mass of the gel composition to be between 50 and 250 g, most often between 100 and 200 g.

Other solid gel compositions are known, for example, those based on d-limonene, solidified by the addition of sodium stearate. Such gels typically contain a greater amount of fragrance than conventional hydrogels, for example, up to a maximum of 15% w/w perfume base. Whilst the d-limonene also contributes to the freshening effect of the product, d-limonene imparts a citrus character to the odour of the product thereby restricting somewhat the range of odours that can be delivered to the atmosphere. Furthermore, d-limonene based gels suffer from the disadvantage that d-limonene is aggressive towards plastics typically used for packaging air fresheners, especially plastics such as polyethylene and polypropylene. Therefore, it is necessary for such gels to be packaged in metal cans.

Attempts have been made to produce improved fragranced gels. Thus, U.S. Pat. No. 5,780,527 describes an anhydrous gel element formed by reacting, in the presence of a perfume base, a liquid polymeric material with a cross-linking agent. The rigid, dry and transparent gel element that is thus formed can contain a large proportion of perfume base, of up to 90% w/w based on the total composition. This last feature is of particular importance since devices incorporating the gel element can be used in the form of discreet articles of small dimensions, notwithstanding of good effectiveness. However, whilst the gels make good air fresheners, they are difficult to process. The process described in U.S. Pat. No. 5,780,527 is a cold mix process. Such a process is disadvantageous in that, inter alia, since the gels are formed by chemical cross-linking, once the gel is set it is not reversible. Furthermore, there are restrictions on the fragrances which can suitably be used with cross-linking agents.

We have found that it is also possible to form an anhydrous gel element by dissolving a cellulose ester at an elevated temperature, in the presence of a perfume base. Gels derived from the process of the present invention can contain a large proportion of perfume base, up to 80% w/w or more, based on the total composition. Thus, such gels possess the high loading advantages of some of the prior art gels but are also more easily processed and/or processable.

Thus, according to the invention we provide a gel comprising from 3 to 80% w/w of perfume base, characterised in that the gel is a substantially anhydrous and thermally reversible gel.

A wide variety of materials may be suitable for the gel of the invention. However, especially preferred are cellulose esters.

It may be disadvantageous to process the gel element of the invention at too high a temperature, as this can be deleterious to the perfume base. Thus, preferably the gels of the invention may be manufactured by dissolving, for example, the cellulose ester(s) in a perfume base at a temperature of up to 130° C., preferably from 60° C. e.g. 60 to 100° C.

Cellulose esters are prepared from cellulose, obtained from wood pulp or cotton linters. Esterification of the cellulose may be performed by mixing the cellulose with the appropriate organic acids, acid anhydrides, and catalysts, thereby converting the cellulose to a cellulose triester. Ester hydrolysis may then be performed by adding a water-acid mixture to the cellulose triester. The solution is filtered and then water is added to precipitate the desired cellulose ester. This is then washed, dewatered, dried and screened to produce the final product. There are various commercially available cellulose esters, for example those marketed by the Eastman Chemical Company. Preferred cellulose esters are cellulose acetate (CA) esters, and mixed cellulose esters, for example, cellulose acetate alkanoate esters, such as, It may be disadvantageous to process the gel of the invention at too high a temperature, as this can be deleterious to the perfume base

Thus, according to the invention we provide a gel element comprising from 3 to 80% w/w of perfume base, characterised in that the gel element comprises a thermally reversible substantially anhydrous gel wherein the gel is a cellulose acetate alkanoate gel.

The cellulose acetate alkanoate used in the gel of the invention is preferably one in which the alkanoate is a C2 to C6 alkanoate, more preferably a C2 to C4 alkanoate. The most preferred cellulose acetate is one selected from the group cellulose acetate propionate (CAP) and cellulose acetate butyrate (CAB) and combinations thereof.

In one embodiment of the invention the anhydrous gel substantially comprises CAP. However, it has been found that CAB is the most useful in the preparation of the gel compositions of the invention. Alternatively, mixtures of CAB and CAP may also be used.

The wide range properties available in CAB, is a consequence of the wide range of butyryl, acetyl and hydroxyl levels. Cellulose esters with a low butyryl content are generally insoluble in most perfume bases, whilst cellulose esters with a very high butyryl content are very soluble in perfume bases, to such an extent that a gel will not form on cooling. Thus, CAB with a “medium” butyryl level, is preferred since it is sufficiently soluble so as to produce a transparent solution with little or no co-solvent, and yet is not so soluble that a gel does not form upon cooling. Thus, a gel with a butyryl level of from 5% to 40% w/w is especially preferred, more preferably from 20% to 40% w/w. Alternatively, a gel with a propionate level of from 5 to 40% w/w may be used, more preferably from 20 to 40% w/w.

Of particular importance in the gels of the present invention is the ratio of the butyryl to acetyl groups in the hydrogel or the propionate to acetyl groups, e.g. the higher alkanoyl to acetyl ratio.

Thus the ratio of higher alkanoyl to acetyl may be within the range of from 1:2 to 50:1 by weight, preferably from 1:1.75 to 17:1 by weight, more preferably from 1.5:1 to 3:1.

Specific cellulose acetate alkanoyls which may be mentioned are the cellulose acetate butyrates, such as CAB 381-20, CAB 381-2.0, or CAB 381-0.5.

The range of cellulose acetates may be extended by the incorporation of a solvent.

In order to improve the solubility of the CAB in the perfume base it is useful to include a co-solvent in the composition. Suitable co-solvents include, but are not limited to, ketones, esters, glycol ethers, and glycol ether esters. The glycol ethers are particularly useful, such as Dowanol PM (propylene glycol monomethyl ether, available from Dow Chemicals), Dowanol DPM (dipropylene glyco monomethyl ether, available from Dow Chemicals) and Dowanol TPM (tripropylene glycol monomethyl ether, available from Dow Chemicals). Dowanol DPM is particularly preferred.

Tables 1 and 2 illustrates the properties of compositions prepared using different. grades of CAB, and the effect of dipropylene glycol monomethyl ether (DPGMME).

Any of the compositions currently used in perfumery. May be used as a perfume base in the gels of the present invention. These can be discreet chemicals, more often however they are mixtures of volatile liquid and/or solid ingredients of natural or synthetic origin. The nature of these ingredients can be found in specialised books of perfumery, e.g. in S. Artander (Perfume and Flavor Chemicals, Montclair N.J., USA 1969). Suitable perfume bases are available commercially from a number of fragrance suppliers.

The gel or gel elements of the invention may be self supporting. Thus, for example, the gel may be absorbed into or onto a porous substrate, including but not limited to ceramic, wood, stone, paper, concrete, an extruded/cast or pressed detergent block (e.g. toilet cleaning block or a soap bar), a sponge, etc.

Alternatively, the gel may be adapted such that it can hold itself on a rough surface, including but not limited to plastics with a rough surface frosted or rough glass, metal wax, etc.

In a yet further alternative, the gel may be able to dissolve controllably through the addition of other materials, including but not limited to enzymes, for example cellulase.

In addition, the gel may includes other materials, including but not limited to surfactants; fillers, such as, chalk, water, etc.; and solvents, such as glycol ethers, etc.

The gels of the invention may be suitable for use as, inter alia, conventional air fresheners. However, one or more conventionally known germicide, bactericide or insecticide materials or any combination thereof may be included in the gel formulation. Thus such gel formulations may be suitable for use as a lavatory cleanser/deodoriser and/or as an insecticide/deodoriser.

Samples were prepared as follows:

(i) Premix the perfume base and DPGMME (where appropriate) in a sealed vessel equipped with a stirrer and heat source.

(ii) Stir vigorously whilst adding the CAB to the vessel.

(iii) Heat the contents with stirring until the CAB dissolves in the perfume/DPGMME mixture.

(iv) Transfer to containers and allow to cool to ambient temperature.

(v) Assess when completely cooled.

The invention will now be described, but shall not be limited, with reference to the accompanying examples.

### EXAMPLE 1

35.0 g of perfume base (apple scent) was mixed with 5.0 g of Dowanol® DPM (available from Dow Chemicals) in a 100 ml sealed vessel, equipped with an overhead stirrer. 10.0 g of CAB-381-0.5 (available from Eastman Chemical Company) was added with vigorous mixing to ensure good dispersion. The degree of mixing was reduced, and the contents of the flask heated to 80° C. After approximately 5 minutes continuous stirring at 80° C. a transparent, viscous liquid was formed. This was allowed to cool to approximately 70° C.

6 g of the mixture was poured into a circular glass dish with a surface area of 25 cm² and allowed to cool to ambient temperature, whereupon a transparent, solid gel was produced. Left in the air the gel lost 4.08 g in 35 days.

### EXAMPLE 2

35.0 g of perfume base (lemon scent) was mixed with 5.0 g of Dowanol® DPM (available from Dow Chemicals) in a 100 ml sealed vessel, equipped with an overhead stirrer. 10.0 g of CAB-381-0.5 (available from Eastman Chemical Company) was added with vigorous mixing to ensure good dispersion. The degree of mixing was reduced, and the contents of the flask heated to 80° C. After approximately 5 minutes continuous stirring at 80° C. a transparent, viscous liquid was formed. This was allowed to cool to approximately 70° C.

6 g of the mixture was poured into a circular glass dish with a surface area of 25 cm² and allowed to cool to ambient temperature, whereupon a transparent, solid gel was produced. Left in the air the gel lost 3.79 g in 30 days.

TABLE 1

| Formulation | Composition % w/w | | | CAB Properties | | | |
|---|---|---|---|---|---|---|---|
| Code | Perfume | CAB | DPGMME² | Acetyl % | Butyryl % | Hydroxyl % | Viscosity/S |
| A | 60 | 20 | 20 | 2 | 52 | 1.8 | 0.2 |
| B | 70 | 20 | 10 | 2 | 52 | 1.8 | 0.2 |
| C | 80 | 20 | 0 | 2 | 52 | 1.8 | 0.2 |
| D | 60 | 20 | 20 | 2 | 46 | 4.8 | 0.3 |
| E | 70 | 20 | 10 | 2 | 46 | 4.8 | 0.3 |
| F | 80 | 20 | 0 | 2 | 46 | 4.8 | 0.3 |
| G | 60 | 20 | 20 | 3 | 50 | 1.7 | 1.9 |
| H | 70 | 20 | 10 | 3 | 50 | 1.7 | 1.9 |
| I | 80 | 20 | 0 | 3 | 50 | 1.7 | 1.9 |
| J | 60 | 20 | 20 | 13.5 | 38 | 1.3 | 0.5 |
| K | 70 | 20 | 10 | 13.5 | 38 | 1.3 | 0.5 |
| L | 80 | 20 | 0 | 13.5 | 38 | 1.3 | 0.5 |
| M | 60 | 20 | 20 | 13.5 | 37 | 1.8 | 20 |
| N | 70 | 20 | 10 | 13.5 | 37 | 1.8 | 20 |
| O | 80 | 20 | 0 | 13.5 | 37 | 1.8 | 20 |
| P | 60 | 20 | 20 | 17.5 | 32 | 1.3 | 0.1 |
| Q | 70 | 20 | 10 | 17.5 | 32 | 1.3 | 0.1 |
| R | 80 | 20 | 0 | 17.5 | 32 | 1.3 | 0.1 |

¹Perfume base = Lavender scent
²DPGMME = Dipropylene glycol monomethyl ether

TABLE 2

| Formulation | |
|---|---|
| Code | Gel Description |
| A | Transparent. Does not solidify upon cooling. |
| B | Transparent. Does not solidify upon cooling. |
| C | Transparent. Does not solidify upon cooling. |
| D | Transparent. Does not solidify upon cooling. |
| E | Transparent. Does not solidify upon cooling. |
| F | Transparent. Does not solidify upon cooling. |
| G | Transparent. Does not solidify upon cooling. |
| H | Transparent. Does not solidify upon cooling. |
| I | Transparent. Does not solidify upon cooling. |
| J | Transparent. Forms rubber-like solid gel upon cooling. |
| K | Transparent. Forms rubber-like solid gel upon cooling. |
| L | Slightly cloudy. Forms rubber-like solid gel upon cooling. |
| M | Transparent. Forms solid gel upon cooling. |
| N | Transparent. Forms solid gel upon cooling. |
| O | Slightly cloudy. Forms solid gel upon cooling. Some |
| | syneresis. |
| P | Very slightly cloudy. Forms rubber-like solid gel upon |
| | cooling. |
| Q | Slightly cloudy. Forms rubber-like solid gel upon cooling. |
| | Some syneresis. |
| R | Insoluble. Does not mix. |

1. A gel comprising from 3 to 80% w/w of perfume base, wherein the gel comprises a substantially anhydrous thermally reversible gel.

2. The gel of claim 1 wherein the gel is produced at a temperature of up to approximately 130° C.

3. The gel of claim 2 wherein the gel is produced at a temperature of at least 60° C.

4. The gel of claim 3 wherein the gel produced at a temperature of from 60 to 100° C.

5. The gel of claim 1 wherein the gel comprises one or more cellulose esters.

6. The gel of claim 5 wherein one or more of the cellulose esters is a cellulose acetate ester.

7. The gel of claim 6 wherein one or more of the cellulose acetate esters is a mixed cellulose ester.

8. The gel of claim 7 wherein the cellulose acetate ester is a cellulose acetate alkanoate ester.

9. The gel of claim 8 wherein the cellulose acetate alkanoate ester is one in which the alkanoate is a C₂ to C₆ alkanoate.

10. The gel of claim 9 wherein the cellulose acetate alkanoate ester is one in which the alkanoate is a C₂ to C₄ alkanoate.

11. The gel of claim 10 wherein the cellulose acetate alkanoate is one selected from the group consisting of cellulose acetate propionate and cellulose acetate butyrate and combinations thereof.

12. The gel of claim 11 wherein the anhydrous gel comprises cellulose acetate propionate.

13. The gel of claim 11 wherein the anhydrous gel comprises cellulose acetate butyrate.

14. (canceled)

15. The gel of claim 11 wherein the alkanoate level is from 5% to 40% w/w.

16. The gel of claim 15 wherein the alkanoate level is a butyryl level.

17. The gel of claim 15 wherein the alkanoate level is a propionate level.

18. The gel of claim 9 wherein the ratio of alkanoate to acetyl is within the range of from 1:2 to 50:1 by weight.

19. The gel of claim 18 wherein the ratio of alkanoate to acetyl is within the range of from 1:1.75 to 17:1 by weight.

20. The gel of claim 19 wherein the ratio of alkanoate to acetyl is within the range of from 1.5:1 to 3:1.

21. The gel of claim 20 wherein the cellulose acetate alkanoate is a cellulose acetate butyrate.

22. (canceled)

23. The gel of claim 6 further comprising a solvent.

24. The gel of claim 23 wherein the solvent is selected from the group consisting of, ketones, esters, glycol ethers, and glycol ether esters.

25. The gel of claim 24 wherein the solvent is a glycol ether.

26. The gel of claim 25 wherein the glycol ether is selected from the group consisting of propylene glycol monomethyl ether dipropylene glycol monomethyl ether, and tripropylene glycol monomethyl ether.

27. The gel of claim 1 wherein the gel is adapted to controllably dissolve.

28. The gel of claim 27 wherein an enzyme is incorporated into the gel.

29. The gel of claim 28 wherein the enzyme is cellulase.

30. The gel of claim 1 wherein the gel further comprises a surfactant, a filler, a solvent, or a combination of any of the aforementioned.

31. The gel of claim 30 wherein the filler is selected from the group consisting of chalk and water.

32. The gel of claim 30 wherein the solvent is a glycol ether.

33. A gel element comprising from 3 to 80% w/w of perfume base, wherein the gel element comprises a substantially anhydrous thermally reversible gel wherein the gel is a cellulose acetate alkanoate gel.

34. The gel element of claim 33 wherein one or more germicide, bactericide, anti-bacterial, deodorising, insecticide, insect attractant, insect repellent materials, or any combination thereof are included in the gel formulation.

35. A gel element wherein the element is held in position by a shrink fit process.

36. The gel element of claim 35 wherein the shrink fit process comprises shrinking the gel around a single locator.

37. The gel element of claim 35 wherein the shrink fit process comprises tensioning the gel element.

38. The gel element of claim 37 wherein the gel element is tensioned between at least a pair of locators.

39. The gel element of claim 33 wherein the gel element is self supporting.

40. The gel element of claim 39 wherein the gel is absorbed into or onto a porous substrate.

41. The gel element of claim 40 wherein the porous substrate is selected form one or more materials selected from the group consisting of ceramic, wood, stone, paper, concrete, an extruded/cast, pressed detergent block, and a sponge.

42. The gel element of claim 39 wherein the gel is adapted to hold rough surface.

43. The gel element of claim 42 wherein the rough surface is selected from the group consisting of frosted glass, rough glass, and metal wax.

44. An air freshener comprising the gel element of claim 33.

45. A lavatory cleanser/deodoriser comprising the gel element of claim 33.

46. An insecticide, insect attractant, insect repellent, deodoriser or germ killer comprising the gel element of claim 33.

47. A process of manufacturing the gel of claim 1 which comprises producing the anhydrous gel at a temperature of up to 130° C.

48. The process of claim 47 wherein the production is carried out at a temperature of at least 60° C.

49. The process of claim 48 wherein the production is carried out at a temperature of from 60 to 100° C.

50. (canceled)
